(19) United States
(12) Patent Application Publication
(10) Pub. No.: US 2009/0148941 A1
(43) Pub. Date: Jun. 11, 2009

(54) Disposable mini-bioreactor device and method

(76) Inventors: Peter Florez, Greenbrae, CA (US); Shun Luo, Irvine, CA (US); Louis Cheung, Alameda, CA (US); Kimberley Florez, Greenbrae, CA (US)

Correspondence Address: QUINE INTELLECTUAL PROPERTY LAW GROUP, P.C., P O BOX 458, ALAMEDA, CA, 94501, US

(21) Appl. No.: 12/221,252

(22) Filed: Jul. 30, 2008

Related U.S. Application Data
(60) Provisional application No. 60/962,723, filed on Jul. 30, 2007.

(51) Int. Cl. C12N 5/02; C12M 1/24; C12N 1/00; B65D 41/04; B65D 41/20; B65D 51/16

(52) U.S. Cl. 435/325; 435/297.1; 435/420; 435/243; 215/248; 215/261; 215/329

(57) ABSTRACT

This invention provides cylindrical cell culture tubes with a cap having both a septum and gas exchange membranes. The culture tubes can be used to inoculate media, culture cells, harvest cells and store cells in the same container with reduced risk of contamination, while facilitating automated handling.

### CROSS-REFERENCE TO RELATED APPLICATIONS

This application claims priority to and benefit of a prior U.S. Provisional Application No. 60/962,723 filed Jul. 30, 2007, and titled “Disposable Mini-Bioreactor Device and Method” by Peter Florez, et al. The full disclosure of the prior application is incorporated herein by reference.

### FIELD OF THE INVENTION

Embodiments of the present invention are directed to methods and systems for processing biological materials, and more particularly, to disposable components/systems for processing biological materials in a highly automated and rapid manner while maintaining high cell viability, throughput and sterility. In particular, the invention in an aspect can be directed to small disposable bioreactors with septa for insertion and removal of samples, and a gas permeable membrane for gas exchange with the external environment.

### BACKGROUND OF THE INVENTION

Cell culture flasks, culture tubes, and bottles range from cotton stoppered Erlenmeyer flasks to computer controlled large scale bioreactors. Experimentation to optimize culture parameters can be done in relatively small containers to save time and expense before scale-up. However, currently available small scale cell culture containers suffer from difficult handling, incompatibility with readily available robotic handling systems, unacceptable rates of contamination and poor gas exchange.

Existing technology in the form of vented and un-vented standard 50 ml centrifuge tubes used to support current cell culture media optimization testing, transfection and other cell banking and process development applications and methods is unable to support near-future, very-fast methods of high throughput testing. This is because these products consisting primarily of standard non-vented and vented centrifuge tubes (including TPP, Switzerland) “disposable bioreactors” with their “vent only” design requires that caps must be manually removed if any manipulation of the cell culture or bio-solutions contained within is desired during testing and/or screening. Current standard vented centrifuge tubes (e.g., “disposable bioreactor” devices) have this serious limitation in the requirement to open a screw-cap to access the interior. Cap removal for inoculation and sampling increase the amount of labor and time required to run experiments or analyses. Sterility and speed are compromised with currently available technology, which can not effectively interact with automated high-throughput processing equipment.

One culture container that addresses some of these issues is the cell cultivating flask described by Lacey in U.S. Pat. No. 7,078,228. Lacey describes a 96-well sized rectangular culture flask, including a gas exchange membrane and access septum. The culture system includes a top plate and a rigid bottom tray of substantially rectangular shape connected by side and end walls, the body of the flask has imparted therein a gas permeable membrane that will allow the free flow of gases between the cell culture chamber and the external environment. The flask body also includes a sealed septum that will allow access to the cell growth chamber by means of a needle or cannula. The system is not well designed for suspension culture and can be difficult to process robotically. For example, the Lacey system requires transfers to additional containers for standard processing steps, such as centrifugation. The location of septa and membranes prevents full use of the container volume. The Lacey system remains fairly complex and prohibitively expensive for some applications.

In view of the above, a need exists for a cost effective disposable bioreactor system that is vented, has a septum and is entirely disposable. It would be desirable to have a bioreactor system well adapted to suspension culture. Benefits could also be realized through a culture system based on containers suitable for robotic applications. The present invention provides these and other features that will be apparent upon review of the following.

### SUMMARY OF THE INVENTION

Embodiments of the present invention address the drawbacks and shortcomings of the prior art in disposable mini bioreactor units and present improved (and optionally disposable) mini-bioreactor systems, disposable container disclosures, and in a preferred application area, mini-bioreactor systems. In particular, embodiments of the present invention provide accessibility, aeration and/or process control and sterility while facilitating rapid testing in conjunction with the use of automated robotic liquid handling equipment or other high-throughout systems.

Cell culture systems utilizing the invention can include, e.g., disposable shaker flasks, media bottles, and media containers comprising approximately the dimensions of a standard 50-ml centrifuge tube and a screw-on cap for closure of the container. The cap can provide a septum and gas permeable membrane, so that culture media can be added or withdrawn through the septum and gasses can be exchanged between an inside and outside of the tube (e.g., an internal environment and an external environment) through the bacterial retentive vent. For example, the present invention can include a disposable mini bioreactor system comprised of: a disposable container for housing bio-solutions for processing, the disposable cap including a single slitted or non-slitted septum port centrally located, as well as up to six, but no less than at least one, gas venting ports. The container can be a standard centrifuge tube or disposable shaker flask. Optionally, the container can be a custom tube incorporating molded turbulence promoting baffles on the bottom sidewall such that, when combined with agitation on a rotational incubator shaker of appropriate amplitude, produces mixing sufficient to mimic that of large scale systems. The systems of the claimed invention when used, for example, in conjunction with robotic automated laboratory systems, support automated high-throughput screening testing, research, screening, cloning research, cell line development, process and cell culture optimization parameters, and sampling. The present invention also includes novel caps for containers. The caps have a body structure with an outer surface, a first inner surface configured to interact with a top edge of the cylindrical container, and a second inner surface; two or more ports positioned within an area defined by the second inner surface and traversing the body structure; and one or more of a self-sealing septum and a gas permeable membrane positioned adjacent to the second inner surface and proximal to the two or more ports. In one preferred embodiment, the caps have central port sealed with the septum and one or more vent ports covered with the gas permeable membrane and radially-distributed with respect to the central port. Optionally, the cap further includes a novel retainer ring configured to position the self-sealing septum and/or the gas permeable membrane proximal to the ports.

The bioreactor can be fabricated with any appropriate material, such as a polypropylene/polysulfone, polyethersulfone, ABS/polycarbonate, a polyacrylic plastic tube container, and/or the like. The disposable septum-membrane-cap of the reactor may be fabricated by modifying standard caps, such as found in standard 50-milliliter disposable centrifuge tubes, disposable shaker flasks, or disposable media bottles. The caps can be newly molded from appropriate material, such as polypropylene or polyethylene. The container (bioreactor reservoir) can include two or three baffles, e.g., at least 1″ in length (vertically) and 2-6 mm in depth (radially).

The container tube can be closed with a specially modified or custom molded cap of polyethylene, ABS, poly acrylic or polypropylene, and/or the like. The cap can be configured as, e.g., a centrifuge cap modified with openings and a separate molded retainer-ring, ultrasonically welded to secure hydrophobic and/or oleophobic membrane(s) and a slitted or non-slitted medical grade silicone septum. The septum can be initially non-perforated, or may be slitted in a straight, “H”, symmetrical “Y”, cross, or star pattern (see FIG. 5).

Accessories to the bioreactor system can include means to agitate, automate, control temperatures, control gasses, control pH, and/or the like. For example, the bioreactor can be mixed by a shaker external to the container, e.g., by placing the container into a shaker holder adapted to functionally receive the container. The cell culture environmental conditions of the bioreactor can be controlled by an incubator, e.g., by placing the container into a cell culture incubator with, e.g., temperature and gas (e.g., CO₂) control systems. The bioreactor can be retained or manipulated in a clean and controlled environment, such as, e.g., a laminar flow hood or clean room. A high throughput screening system can track, incubate, inoculate, suspend, feed, split, centrifuge, sample, and/or analyze cell cultures grown in the containers, e.g., for optimization of cell media formulations; testing of cell media additives for stimulating cell growth and/or protein expression; high throughput screening of cell and cell product processing parameters; high throughput (HTP) process development, and/or the like.

The bioreactors of the invention can be used to culture many different cell lines including, e.g., mammalian cells, microbial cells, plant cells, yeast, insect cells, CHO cells, 293 cells, hybridomas, BHK cells, Vero cells, MCBK cells, NSO cells, bacterial cells and/or the like. The cells can be subject to rapid high throughput screening using systems of the invention.

A bioreactor system of the invention can include an automated laboratory liquid handling system applicable to high throughput screening for cell culture process development. For example, the bioreactor system can allow an automated laboratory liquid handling system to be applied to high throughput screening for media optimization; to high throughput screening for cell line development; to high throughput screening for cloning screening; to high throughput screening for bioreactor process optimization; to high throughput screening for process characterization; to high throughput screening for validation of these processes, and/or the like.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “media” can include mixtures of media, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

As used herein, the terms “about” or “approximately” refer to a value at or near the cited value. For example a value within 25%, 10% or 5% of the cited value would be considered “about” or “approximately” the cited value.

As used herein, a “septum” is a sheet of material extending across an access port in a cap of the invention. A “resilient septum” is a septum made of a material which will rebound, after penetration through the septum at a location with a conduit and removal of the conduit from the septum, so that significant amounts (under the conditions of use) of water will not leak through the septum. For example, a preferred resilient septum will not leak water through more than 1 ml per minute at a location after penetration and removal of a 1 mm diameter pin while holding back water at a pressure differential of 0.05 pounds per square inch (e.g., holding back a head of approximately 2 inches of water). That is, resilient septa of the invention can functionally retain 2 inches of media after a puncture and inversion of the container. The septum, after penetration and removal of a needle or cannula, can be rebound to provide a hermetic seal, or to provide a functional seal that, although not perfectly hermetic, prevents release of liquids or cells from the container.

As used herein, the term “gas permeable membrane” refers to a porous membrane that (such as a filter) that allows gasses to be significantly exchanged from one side to the other across the membrane. For example, the gas permeable membranes are not impermeable to gasses. Functional gas permeable membranes of the invention allow adequate gas exchange between the interior of the reactors and the external atmosphere to provide adequate respiration through the membrane for cell growth.

As used herein, the term “cylindrical” refers to containers having at least one cross-section defined in part by an arc or rounded shape (e.g., at least a portion of a cross-section is axially or radially symmetrical). The cylindrical containers of the claimed invention include “ovoid” and “flat-sided” as well as “round” tubular shapes. The cylindrical containers of the claimed invention also include containers that taper in diameter, e.g. from top to bottom, or from bottom to top, and containers that are not cylindrical their entire length (e.g., containers that have multiple openings for accessing an interior volume of the container.

### BRIEF DESCRIPTION OF THE DRAWINGS

FIG. 1 is a schematic diagram of typical standard disposable 50-ml centrifuge tubes.

FIG. 2 is a schematic diagram of an exemplary bioreactor system of the invention including a cap with both a septum and gas exchange ports; a flat container bottom end and agitation baffles.

FIG. 3A to 3C shows schematic diagrams of exemplary bioreactor caps of the invention.

FIG. 4 shows a schematic diagram of an exemplary cap assembly with a septum, gas exchange filter and retainer ring.

FIG. 5 shows a schematic diagram of exemplary septum perforation slits for access to the reactor interior.

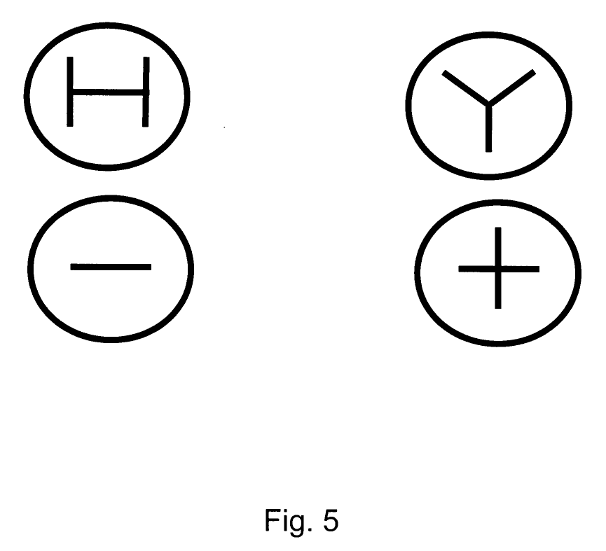

FIG. 6 shows an automated robotic system for handling and processing of, e.g., bioreactor arrays.

FIG. 7 is a figure depicting cell growth profiles generated using TPP tubes versus exemplary bioreactor systems.

### DETAILED DESCRIPTION

The invention includes convenient disposable small-scale bioreactors. The bioreactors, or cell culture tubes, are typically provided in standard dimensions, e.g., of standard commercially available 50-ml centrifuge tubes, for compatibility with centrifuges, shakers, robotic equipment and racks in widespread use. The bioreactors can include features, such as, e.g., a container with cylindrical side walls, a closed bottom end and an open top end. The container can be closed with a screw-on cap, which has a septum and a gas permeable membrane. The membrane can allow gas exchange between an inside of the container and the exterior of the container. The septum can provide a self-sealing access to the inside of the container by insertion of a needle or cannula. Such a system can allow small volume cell culture and large parallel culture studies with a reduced chance of contamination and high suitability for automated processing.

The methods of the invention can be practiced using the bioreactors of the invention to culture cells in media. For example, a method of culturing cells can include providing a cylindrical culture container having a screw-on cap with a gas permeable membrane and a septum. The container can receive a cell culture media and inoculation with cells of interest. The cells can grow and expand in number to produce progeny cells. Media and/or cells can be harvested by drawing them up through a conduit inserted aseptically through the cap septum and into the media.

### Bioreactor Tubes

Bioreactor tubes and containers of the invention can include a tube or other enclosable container coverable with a cap having ports covered with a resealable septum and one or more gas permeable membranes. The inside of the tube (container) can be configured for various culture conditions, such as, for suspension culture or culture of cells in a lawn. The tubes can be filled with appropriate cell culture media and inoculated with cells of interest for growth and study. The tubes can be held in an incubator and manually or robotically manipulated.

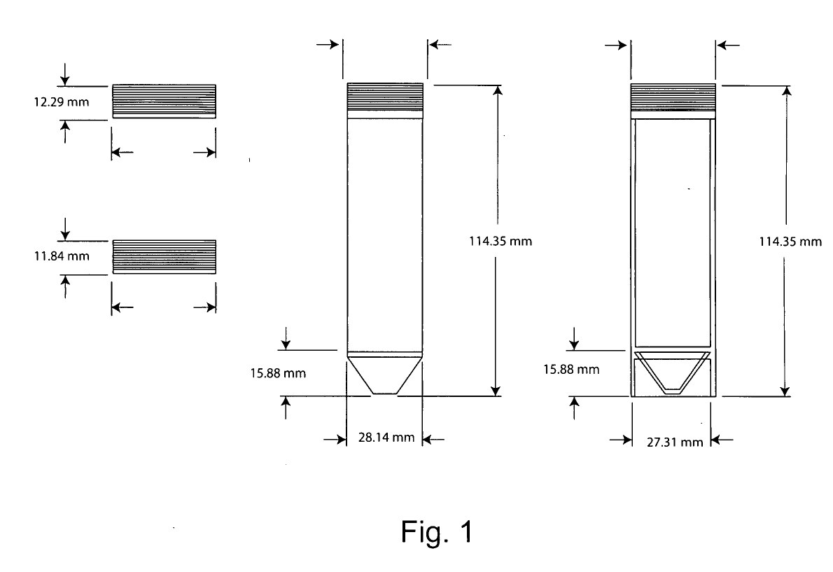

A common laboratory tube is the standard 50-ml capped test-tube, as shown, e.g., in FIG. 1. The typical dimensions are standardized across many vendors (e.g., CORNING, VWR, BD FALCON, etc.) and hardware of every kind is available for handling, storing and processing these standard containers. Although the tubes are designed for use as centrifuge tubes, such containers can be sterilized and used for cell culture. However, no significant gas exchange occurs through the closed tubes and gas exchange can be poor with the caps loosened. Spillage past loosened caps can lead to contamination through the loosened caps. Loosened caps can shake free in shakers. Manual manipulation (e.g., cap removal steps) during processing, including inoculation, splitting and harvest with the cap removed can greatly increase the likelihood of contamination and require the use of a laminar flow hood with a HEPA filter. Automated processing of the standard tubes can be difficult because of the poor robotic access through the solid screw-cap closure.

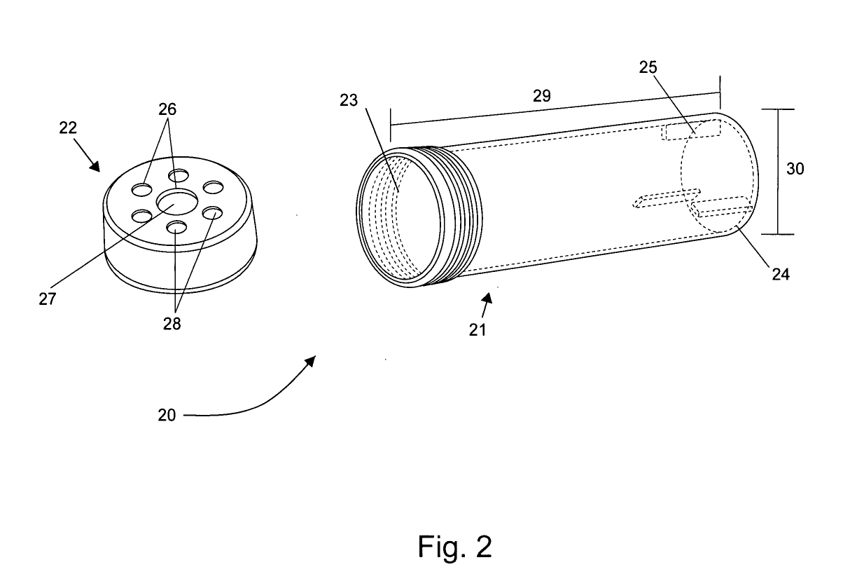

The present invention has unique combinations of features that allow a standard size tube to be used effectively as a cell culture reactor. For example, as shown in FIG. 2, the cell culture system 20 of the present invention can include a cylindrical container 21 and a cap 22 adapted to cover the top opening 23 of the container. The container can have a flat bottom 24 and/or baffles 25. The cap can have ports 26 spanned by septum 27 and/or gas permeable membranes 28.

Systems of the invention can include accessory or support subsystems to provide, e.g., storage, incubation, transport and/or processing of the culture tubes. For example, the systems can include culture media, incubators, sampling conduits, robotic processors, shakers, and the like.

### Reactor System Containers

The containers of the invention can be essentially cylindrical tubes closed at one end (bottom) and open at the other end (top). The open end can be adapted, e.g., with a snap-seal ridge or threads, to receive a closure cap. In preferred embodiments, the container has the basic dimensions of a standard 50-ml “30×115” disposable plastic centrifuge tube, e.g., as shown in FIG. 1. However, the standard configuration can be modified, e.g., with a flat bottom, turbulence baffles, cell attachment surfaces, etc., as required for a particular cell culture. In certain embodiments, the container can be a shaker flask, media bottle, culture flask or centrifuge tube. In some embodiments, the container structure be shaped as a stir flask, T-flask, media flask, shaker flask, or other functional media container shape known in the art.

In one preferred embodiment (see FIG. 2), the containers can have a length 29 ranging from about 110 mm to about 120 mm, from about 112 mm to about 117 mm, from about 113 to about 116 mm, or about 115 mm. The containers can have a diameter 30 ranging from about 31 mm to about 26 mm, from about 30 mm to about 27 mm, from about 29 mm to about 28 mm. Preferably the container has a length of about 115 mm and a diameter of about 29 mm. In many embodiments, the essentially cylindrical container tapers slightly from top to bottom, e.g., to facilitate manual or robotic holding without slipping, and to facilitate insertion and removal of the container to and from holders, such as racks and centrifuges. For example, the diameter of the container can taper in diameter from top to bottom about 2 mm, 1 mm, 0.5 mm, or 0 mm. In one embodiment, the container tapers from 29 mm at the top end to 28 mm at the bottom end.

The containers can be fabricated from any suitable material. For example, the containers can be made of a metal, glass or plastic. In preferred embodiments, the container is disposable, and/or made of polyethylene, high density polyethylene (HDPE), acrylonitrile butadiene styrene (ABS), poly acrylic, polystyrene, or polypropylene, and/or the like.

The bottom end of the container can be flat and squared to the walls, have a conical shape, or have a skirt for free standing. The flat bottom can provide an inside surface for growth of cells that require contact or attachment for growth. A conical bottom can allow cultured cells to be concentrated in a small area, e.g., by centrifugation.

One or more optional baffles can extend radially inward from the walls, and are positioned, for example, at or near the bottom of the container. Such baffles can help generate turbulence in media when the container is moved, e.g., in a shaker. The increased turbulence can help maintain cells and other particles in suspension in the media. In preferred embodiments, the containers include 2 to 6 baffles, and optionally 4 baffles or 3 baffles. The baffles can have a length ranging from 100 mm to about 5 mm, from about 50 mm to about 10 mm, or from about 30 mm to about 20 mm. The baffles can extend inward (preferably radially) a distance of about 2 mm to about 10 mm, or from about 3 mm to about 7 mm. In a preferred embodiment, the claimed containers comprise three baffles arranged with radial symmetry along a cylindrical wall of the container, and have lengths of 20 mm and inwardly-extending heights of 4 mm. In some embodiments, the baffles are separately manufactured components that are inserted into the containers (see, for example, baffles 25 depicted in FIG. 2). In an alternative embodiment, the baffles are formed from the sides or walls of the container, e.g., by heating or otherwise deforming the wall portion of the container.

The inside surface of the container can optionally include a coating of material conducive to cell attachment. This can aid in culture of cells that require contact or attachment for optimal growth. For example, the inner walls and/or bottom end surface can have a coating of a protein or other biopolymer, such as fibronectin.

### Container Caps

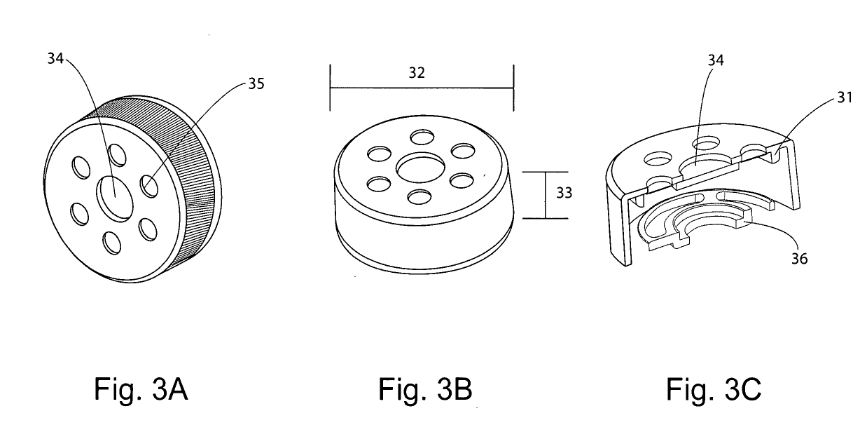

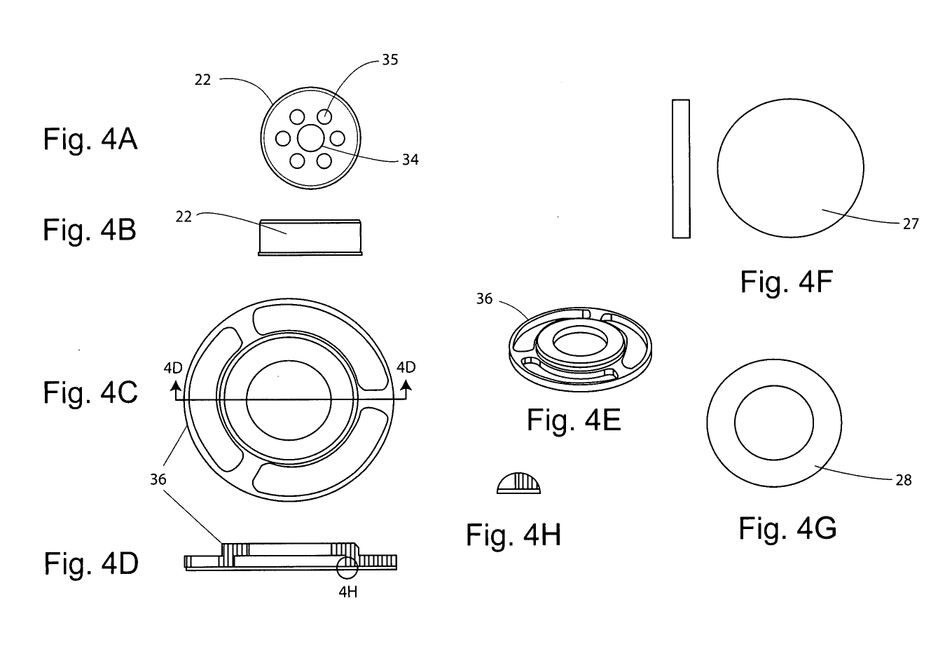

Exemplary caps of the claimed invention are depicted in FIGS. 2 through 4. The caps comprise a body structure having an outer surface, a first inner surface configured to interact with a top edge of the cylindrical container, and a second inner surface, and two or more ports (vent ports) positioned within an area defined by the second inner surface and traversing the body structure. Optionally, the caps further include a self-sealing septum and a gas permeable membrane positioned proximal to the two or more ports. The caps used to cover the top openings of the containers in the inventive systems can be any suitable to cover the containers and to function in structural support of desired septa, membranes, retainers, seals, and the like. In many embodiments, the caps are standard disposable 50-ml centrifuge tube caps modified with ports for mounting of septa, and/or gas exchange membranes, e.g., as shown in FIG. 3A and FIG. 4.

The caps of the culture systems can have an outer surface, one or more inner surfaces, and dimensions suitable to act as a functional closure to the containers of the invention. Typically, the caps snap or threadably fit over the top opening of the container. Alternately, the caps could seal at the top edge of the container or at an inner surface of the container. In some embodiments, the upper (second) inner surface of the cap can include a ridge or compression seal 31 that contacts and interacts with the top edge of the container to form a hermetic of water-proof seal. The caps typically have a width (diameter) 32 ranging from about 40 mm to about 30 mm, or from about 38 mm to about 33 mm; and a height 33 ranging from about 6 mm to about 15 mm, from about 8 mm to about 13 mm, or from about 10 mm to about 12 mm. In a preferred embodiment, the cap has an outer diameter of about 35 mm and a height of about 12 mm.

The caps can include ports (e.g., holes through the plastic cap body) that can functionally accommodate the septa and/or gas exchange membranes of the invention. The septa or membranes can traverse the port to functionally expose a side to the external environment, e.g., for cooperation with external manipulations of gas exchanges. The ports can be arranged in any number, in any size, and in any suitable pattern. Typically, the cap includes at least one port traversed with an access septum, and one or more ports for gas exchange. Typically, the area afforded to gas exchange is greater than the area afforded to septa access. In one embodiment, as shown in FIGS. 3A through 3C, port 34 provided for the septa is located central to ports 35 provided for gas exchange membranes. In preferred embodiments, the port provided for septa has a diameter (or width) ranging from 1 mm to 10 mm, from 2 mm to 8 mm or about 5 mm. In preferred embodiments, there is a single port for multiple use septa. Optionally, there can be multiple ports for access to one or more septa. In preferred embodiments, the total port area for gas exchange membranes ranges from about 4.5 cm² to about 1 cm², about 3 cm² to about 1.5 cm², or about 2.5 cm² to about 2 cm². In preferred embodiments, there are multiple ports for gas exchange, e.g., 6 radially arranged ports, 4 ports, or 3 ports. Optionally, the cap can include a single gas exchange port, e.g., aside a single smaller access septa port.

Septa function to allow resealable penetration by a conduit. For example, septa can be a membranous resilient material that can be pierced, or have a slit, to accommodate penetration of conduit, such as, e.g., a needle, pipette or cannula. The resealable septa can resiliently close around the point of penetration, e.g., so that liquids in contact with the point will not readily flow through the point after the conduit is removed. Typically, the septa are fabricated from a sheet or plug of resilient polymer, such as, e.g., natural or synthetic rubber, silicone rubber (preferably class VI medical-grade silicone rubber), a thermoplastic elastomer (TPE), or any other resilient resealing polymeric material. Exemplary TPEs for use in the caps and systems of the claimed invention include polymeric block copolymers comprising hydrogenated styrene/isoprene-butadiene/styrene, such as C-Flex® (Consolidated Polymer Technologies, Inc, Clearwater, Fla.), and crosslinked polymers of ethylene propylenediene M-class (EPDM) rubber and polypropylene, such as Santoprene™ (Monsanto, ST. Louis, Mo.). In many embodiments, the septa are uniform sheets that can be penetrated, e.g., by a needle, at any point to form a tear, point hole, or small slit that rebounds to close when the needle is withdrawn. Optionally, as shown in FIG. 4, a slit of varying configurations (straight, H-shaped, Y-shaped, star-shaped, etc) can be preformed in the septum membrane. The preformed slits can be cut clear through the membrane or through a substantial portion of the membrane.

Gas exchange membranes are membranes that allow exchange of gases from one side to the other. Typically, the membranes are porous with channels extending through the membrane. Typically, the pores or channels are micro-scale, or nano-scale. In preferred embodiments, the gas exchange membranes are filters with effective pore sizes of about 10 μm, 8 μm, 5 μm, 3 μm, 1 μm, 0.8 μm, 0.6 μm, 0.45 μm, 0.2 μm, 0.1 μm, or less. Typically, the membranes are thin, e.g., 25 μm to 500 μm, 50 μm to 250 μm, or about 100 μm in. In some preferred embodiments, the membranes are hydrophobic so they do not absorb liquid media on contact, thus not becoming occluded on contact with the media. Optionally, the membranes of the claimed invention comprise oleophobic material(s) including, but not limited to, acrylic copolymers; the oleophobic membranes have a greater resistance to wetting with low surface tension fluids. Exemplary membranes include hydrophobic porous membranes, porous polytetrafluoroethylene (PTFE) membranes, porous polyvinylidene fluoride (PVDF) membranes, acrylic co-polymer membranes, porous polypropylene membranes, and/or the like.

The septa and/or membranes can be functionally sealed or mounted across the ports. For example, the septa and/or membranes can be welded (e.g., with heat) to the first and/or second inner surface of the cap, across the ports. The septa and/or membranes can be ultrasonically welded, glued, wedged, compression fitted, held in place with a retainer structure, and/or the like. Exemplary retainer rings of the claimed invention are depicted in FIG. 3C and FIG. 4, panels 4C, 4D, 4E and 4H. In one exemplary embodiment, as shown in FIG. 3C and FIG. 4, retainer ring structure 36 is employed to mount septum disk 27 across septum port 34 and to mount a gas membrane ring (e.g., filter 28) across radially distributed gas exchange ports 35. Retainer ring 36, in turn, can be functionally mounted to the cap, e.g., with a compression fit against an inner surface of the cap or the cap compression seal ridge 31.

FIG. 4, panels A through H depict embodiments and exemplary dimensions of the cap components of the claimed invention. Panels 4A and 4B provide top and side views of cap 22; panels 4C, 4D, 4E and 4H depict a top, side, perspective, and detail views of retainer ring 36; and panels 4F and 4G provide top and side views of the septum 27 and filter 28 components, respectively.

### Culture Media/Cells

The bioreactor systems are suitable for culture of many types of cells. For example, the systems can be used to culture plant cells, bacteria, viruses, eukaryotic cells, primary cell cultures, continuous cell lines, and/or the like. The bioreactor containers of the invention can be used to culture many different cell lines including, e.g., mammalian cells, microbial cells, insect cells, CHO cells, 293 cells, hybridomas, BHK cells, Vero cells, MCBK cells, NSO cells, bacterial cells, and/or the like.

The systems can include media appropriate to the cells for culture. For example, the containers can hold media for culture of cells, including types of growth media, nutrient media, minimal media, selective media, differential media, transport media, enriched media, Ames medium, basal media eagle (BME), click's medium, Dulbecco's modified Eagle's media (DMEM), Dulbecco's modified Eagle's medium/Ham's nutrient mixture F-12, Dulbecco's phosphate buffered saline, Earle's balanced salts, Glasgow minimum essential media, Grace's insect media, Hank's balanced salts, Iscove's modified Dulbecco's media (IMDM), IPL-41 insect medium, L-15 media, M2 and M16 media, McCoy's 5A modified media, MCDB media, medium 199, minimum essential medium Eagle (MEM), NCTC media, HAM F-10, HAM F-12, RPMI-1640 media, Schneider's insect media, Shields and Sang M3 insect media, TC-100 insect medium, TNM-FH insect media, Waymouth medium MB, William's medium E, and/or the like.

### System Accessories

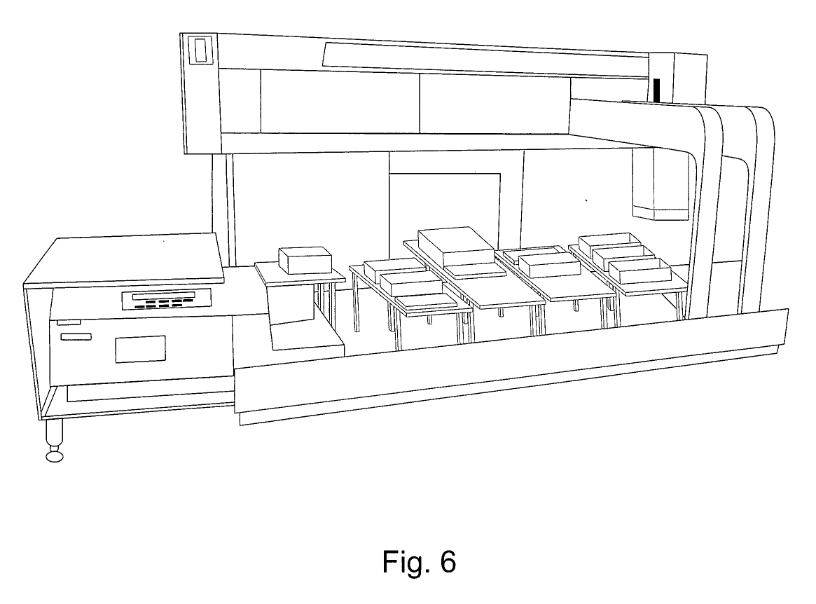

Bioreactor systems of the invention can include accessory sub-systems, e.g., to provide conditions for cell culture and/or to enhance processing efficiency. Such sub-systems can include clean rooms, laminar-flow hoods, incubators, automated tube handling systems, shakers, sensors, and/or the like. For example, the systems can include robotic handling and sampling systems, such as shown in FIG. 6.

### Using Disposable Mini-Reactors

The methods of using the inventive cell culture tubes include provision of the tubes and media, inoculation, and culture. For example, a method of culturing cells can comprise, providing a cylindrical culture container having a screw-on cap with a gas permeable membrane and a septum. Cell culture media is placed inside the container and the media is inoculated with one or more inocula cells of interest. The cells are allowed to grow and divide in the media to produce progeny cells. A conduit can be passed through the septum and into the media, e.g., to add media, or to removing a sample of the progeny cells, inocula cells or media from the culture tube.

The inventive culture tubes had the advantage that they can be handled efficiently by robotic instrumentation. Another significant advantage is the ability to access and vent the culture with minimal risk of contamination, even in environments that are not sterile or aseptic. For example, a closed and previously sterilized bioreactor tube of the invention can be filled with media (e.g., 50 ml to 1 ml, or less) using a sterilized syringe, even without the benefit of a laminar flow hood, and without contamination of the media. For example, when using caps and/or cell culture systems employing a hydrophobic and/or oleophobic membrane component, such as those identified herein, the septa surface can be sanitized by application (e.g., via a wipe, swab or spray) of a sanitizer, such as IPA or alcohol, without wetting and/or clogging the gas exchange pores of the device. Inocula can also be provided through the repeatably usable septa.

For cultures where the cells should remain in suspension, the tubes can be placed on a shaker rack, e.g., in an incubator while the cells are growing. For cultures where the cells are intended to grow on the container bottom surface, the cells can simply be held in the incubator standing vertically in a rack or lying horizontally. For horizontal culture, the ports can be located above the intended level of media. Horizontal cultures can optionally be rotated about a central axis to grow cells attached on the side walls of the container, e.g., periodically dipping the attached cells into media and raising them into the gaseous space.

It is notable that a degree of process control can be achieved in an incubator without compromising the sterile barrier established by the disposable mini bioreactor. For example, gas flow, dissolved oxygen (DO), pH and/or CO₂ can be influenced be controlled by adjusting the rate of gas exchange across the membranes. Optionally, caps with various gas exchange areas and/or with various pore sizes can be selected to obtain a desired exchange rate. Optionally, the gas exchange rate can be adjusted by manually occluding one or more of the gas exchange ports with laboratory tape.

Cultured cells can be harvested without the need to transfer the culture into separate centrifuge tubes. For example, cell suspensions in bioreactor tubes with conical bottom ends can be placed into a swinging bucket centrifuge to harvest the cells in a small pellet at the bottom center of the tube without having to transfer the culture to a new tube. Optionally, cultures of cells attached in lawns can be harvested by aspirating out the media with a cannula through the septum. Rinse solutions and protease solutions can be introduced through the septum to release the cells from the container walls or bottom end surface. For a square end container, the cells can be collected in a small area where the bottom meets the side wall by centrifugation in a fixed angle rotor.

The optionally-disposable mini bioreactor is completely portable and may be used in numerous departments and different locations before, during and after cell culture processing. After irradiation sterilization, the inside of the disposable mini bioreactor may be considered sterile, providing a “sterile enclosure” protecting the contents of the vessel from airborne contaminants outside. The systems of the invention provide the ability to inoculate, culture, harvest and store cells without transfer to another container. The combination of the standardized shape with the septa/membrane aspect of the bioreactors facilitates automated handling and processing.

### EXAMPLES

The following examples are offered to illustrate, but not to limit the claimed invention.

### Example 1

### 50 ml Mini-Reactor Tubes

A disposable mini bioreactor device is presented and is comprised of a disposable plastic vented septum cap and a matching cylindrical container for housing bio-solutions for processing.

One version of the system utilizes pre-existing standard 50 ml centrifuge tubes and caps as shown in FIG. 1. In this first version the standard cap was modified (an array of 7 symmetrical holes punched with a die or laser cut. See FIG. 3A) in such a way to include one centrally located opening or port to accommodate a Class VI medical grade silicone septum. See septum 34 at the inlet-outlet port in cutaway drawing of FIG. 3C. Circularly and evenly spaced round openings provide gas exchange ports below which is mounted an integral 0.2 μm, 0.22 μm, 0.45 μm, or 3 μm membrane vent filter membrane ring 35 (FIGS. 1 and 3).

The septum and membrane ring were each integrally attached via ultrasonic welding to the inside of the cap via an injection-molded retainer-ring indicated in FIG. 3C. One of these rings was welded to the bottom of each cap in such a way as to mechanically bond the centrally located silicone septum and ultrasonically seal the membrane ring.

After modifications and subsequent assembly each bioreactor is sterilized by exposure to e-beam or gamma irradiation. The integrity of the sterile environment is maintained by the filter membrane ring and septum. The design of this device allows for daily manipulation of tube contents for sampling of the cell culture and makes possible the use of an automated robotic liquid handling system as in the FIG. 6.

The tube and septum cap enables the use of automated robotic laboratory liquid handling systems for routine research, screening, process optimization studies and contributes significantly to cost savings, time savings and reduced risk of contamination. Applications for this mini bioreactor technology include, but are not limited to, media optimization, additives screening and testing of media formulations, cell line development/cloning, cell culture optimization, media additives optimization, bioreactor conditions, cell culture optimization, cell banking, cell scale up, transfection, gene therapy, stem cell production and research, protein expression, sampling and process development.

The silicone rubber septum in the current embodiment is centrally located in the cap and has a centrally located slit; this feature allows for a syringe or flat bottom cannula probe to be inserted into the disposable bioreactor tube to add and withdraw liquid components during the cell culture fermentation cycle in such a way as to not allow introduction of contaminants or breach sterility.

A second version of this mini disposable bioreactor will be a completely insert-molded unit featuring a molded cap and molded tube with baffles. See FIG. 1. The septum and filter ring in this second version may be ultrasonically welded or insert molded into the cap but the functionality is identical.

### Example 2

### Cell Culture in Mini-Reactor

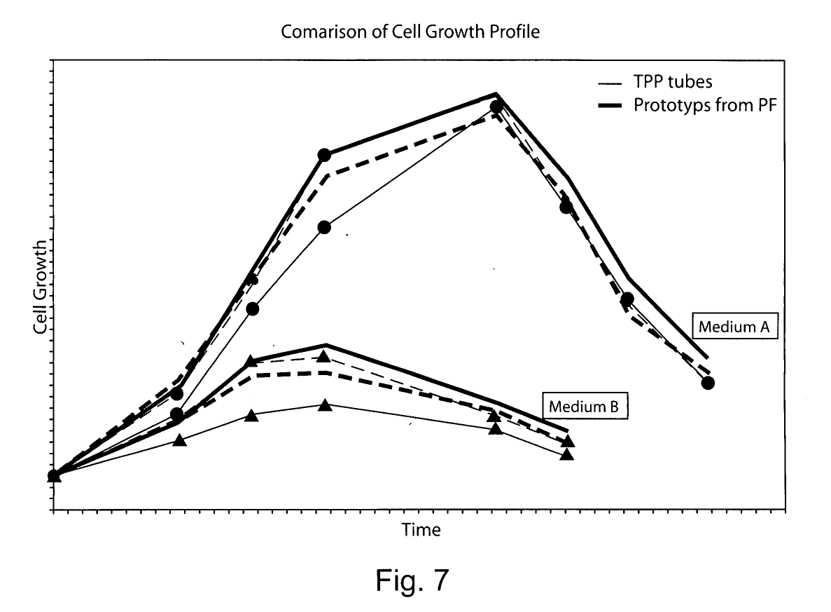

As depicted in FIG. 7, mammalian cells were grown to high density and maintained at high viability, demonstrating the utility of the present invention for culture of eukaryotic cells. In a test run, mammalian cells were inoculated into two different culture media (Media A & Media B) and cultured in a 37 degree Celsius incubator with 5% CO₂ and 80% humidity environment. Experiment was carried out in duplicates and controls were setup in vented centrifuge tubes (TPP) in parallel. Samples were taken for cell count and viability analysis during the experiment. Septa having a preformed opening or slit (typically an “H” slit or “Y” slit) were entered using a 3 mm diameter flat-tipped cannula. No contamination occurred. Results showed that cells were able to grow to high density and maintained at high viability. Cell culture performance, in terms of cell counts and viability, at the present invention was also comparable to the vented centrifuge tubes (TPP). In conclusion, results demonstrated the utility of the present invention, 50-ml bioreactors with a septum and 0.22 micron gas exchange membranes in the cap, for culture of eukaryotic cells.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A cell culture system comprising:
a container comprising a cylindrical side wall, a closed bottom end and an open top end; and,
a cap comprising a septum and a gas permeable membrane, wherein the cap is adapted to close the top end of the container;
wherein the membrane allows gas exchange between an inside of the container and the exterior of the container, and wherein the septum provides a self-sealing access to the inside of the container after penetration with a conduit.

2. The system of claim 1, wherein the container has approximately the length and diameter of a standard 50-ml centrifuge tube.

3. The system of claim 2, wherein the container comprises a length between about 110 mm and about 120 mm, and comprises a diameter between about 30 mm and about 27 mm.

4. The system of claim 1, wherein a floor of the bottom end is perpendicular to the side wall.

5. The system of claim 1, further comprising one or more baffles on the inside at the bottom end.

6. The system of claim 1, wherein the cap further comprises a central port sealed with the septum and further comprises radially distributed vent ports covered with the gas permeable membrane.

7. The system of claim 1, wherein the cap further comprises a retainer ring in the cap functioning to mount the septum in the cap or to mount the membrane in the cap.

8. The system of claim 1, wherein the septum is fabricated from one or more materials selected from the group consisting of: rubber, medical grade rubber, silicone rubber, thermoplastic elastomers (TPEs), and resilient polymers.

9. The system of claim 1, wherein the membrane is fabricated from a material selected from the group consisting of: a hydrophobic porous membrane, an oleophobic porous membrane, acrylic-copolymer, polytetrafluoroethylene (PTFE), polyvinylidene fluoride (PVDF) and polypropylene.

10. A cell culture system comprising:
a media container comprising approximately the dimensions of a standard 50-ml centrifuge tube; and,
a screw-on cap for closure of the container and comprising two or more vent ports, a septum positioned proximal to a first vent port, and a gas permeable membrane positioned proximal to one or more additional vent ports;
whereby culture media can be added or withdrawn through the septum, and gasses can be exchanged between an inside of the container and an outside of the container.

11. The system of claim 10, wherein the cap further comprises a retainer ring in the cap and functioning to mount the septum in the cap or to mount the membrane in the cap.

12. A method of culturing cells, the method comprising:
providing a cylindrical culture container having a screw-on cap, which cap comprises a gas permeable membrane and a septum;
placing cell culture media and one or more inocula cells inside the culture tube;
allowing the one or more cells to divide in the media to produce progeny cells;
passing a conduit through the septum and into the media;
removing a sample of the progeny cells, inocula cells or media from the culture tube through a conduit.

13. The method of claim 12, wherein the cylindrical container has approximately the length and diameter of a standard 50-ml centrifuge tube.

14. The method of claim 12, wherein the cap further comprises a central port sealed with the septum and further comprises radially distributed vent ports covered with the gas permeable membrane.

15. A cap for a cylindrical container, comprising
a body structure having an outer surface, a first inner surface configured to interact with a top edge of the cylindrical container, and a second inner surface;
two or more ports positioned within an area defined by the second inner surface and traversing the body structure; and
a self-sealing septum and a gas permeable membrane positioned proximal to the two or more ports.

16. The cap of claim 15, wherein the two or more ports comprise a central port sealed with the septum and radially-distributed vent ports covered with the gas permeable membrane.

17. The cap of claim 15, wherein the cap further comprises a retainer ring configured to position the self-sealing septum and/or the gas permeable membrane proximal to the ports.

18. The cap of claim 17, wherein the retainer ring further comprises a compression fitting configured for mounting the retainer ring against the first or second inner surface of the cap.

19. The cap of claim 15, wherein the first inner surface of the cap comprising a ridge or compression seal configured to interact with a top edge of the cylindrical container.
